(19) United States
(12) Patent Application Publication
(10) Pub. No.: US 2010/0145432 A1
(43) Pub. Date: Jun. 10, 2010

(54) IMPLANT AND METHOD FOR PRODUCING THE SAME

(76) Inventors: Ullrich Bayer, Admannshagen-Bargeshagen (DE); Baerbel Becher, Rostock (DE); Bernd Block, Rostock (DE); Daniel Lootz, Rostock (DE)

Correspondence Address: GREER, BURNS & CRAIN, 300 S WACKER DR, 25TH FLOOR, CHICAGO, IL, 60606, US

(21) Appl. No.: 12/630,407

(22) Filed: Dec. 3, 2009

(30) Foreign Application Priority Data
Dec. 9, 2008 (DE) 10 2008 054 400.0

(51) Int. Cl. A61F 2/82; B05D 3/10

(52) U.S. Cl. 623/1.15; 427/2.24

(57) ABSTRACT

The present invention describes a method for producing an implant, in particular an intraluminal endoprosthesis, wherein the base material of the body (5) of the implant has biodegradable metallic material, preferably Mg or an Mg alloy. The method comprises the following steps:
a. Provide the body (5) of the implant,
b. Apply a first layer (10), which contains Ca ions and P ions, to at least a part of the surface of the body (5) and
c. Apply a second layer (20), which is at least partially permeable for Ca ions and P ions, such that this at least largely covers the first layer (10).

Furthermore, a corresponding implant is described, in which the degradation behavior can be controlled through the production according to the invention.

### FIELD OF THE INVENTION

The present invention relates to a method for producing an implant, in particular an intraluminal endoprosthesis, as well as a corresponding implant.

### BACKGROUND OF THE INVENTION

A wide variety of medical endoprostheses or implants for various applications are known from the prior art. For purposes of the present invention, implants are understood to be endovascular prostheses, for example, stents, implants used in osteosynthesis, preferably fastening elements for bones, e.g., screws, plates or nails, surgical suture materials, intestinal clamps, vascular clips, prostheses in the area of hard tissue and soft tissue and anchoring elements for electrodes, in particular for pacemakers or defibrillators.

Nowadays stents that are used for treating stenoses (vasoconstrictions) are used particularly frequently as implants. As a body they have a tubular or hollow cylindrical main lattice which is open at both longitudinal ends. The tubular main lattice of an endoprosthesis of this type is inserted into the vessel to be treated and is used for supporting the vessel. Stents have become established in particular for treating vascular diseases. Constricted areas in the vessels can be expanded by the use of stents, so that an increase of lumen results. Although the use of stents or other implants can make it possible to achieve an optimum vessel cross section which is primarily necessary for a successful therapy, the permanent presence of a foreign body of this kind initiates a cascade of microbiological processes which can result in the stent gradually growing shut and in the worst case to a vascular occlusion.

A starting point for resolving those problems is therefore to make the stent or other implants from a biodegradable material as a base material.

The term “biodegradation” refers to hydrolytic, enzymatic and other metabolic degradation processes in the living organism caused mainly by body fluids coming into contact with the biodegradable material of the implant and leading to the gradual dissolution of the structures of the implant containing the biodegradable material. Through this process the implant loses its mechanical integrity at a certain point in time. The term “biocorrosion” is often used synonymously with the term “biodegradation.” The term “bioabsorption” includes subsequent absorption of the degradation products by the living organism.

Materials suitable for the body of biodegradable implants may contain polymers or metals, for example. The body can thereby comprise one or more of these materials. The common feature of these materials is their biodegradability. Examples of suitable polymeric compounds are polymers from the group of cellulose, collagen, albumin, casein, polysaccharides (PSAC), polylactide (PLA), poly-L-lactide (PLLA), polyglycol (PGA), poly-D,L-lactide-co-glycolide (PDLLA-PGA), polyhydroxybutyric acid (PHB), polyhydroxyvaleric acid (PHV), polyalkyl carbonates, polyorthoesters, polyethylene terephtalate (PET), polymalonic acid (PML), polyanhydrides, polyphosphazenes, polyamino acids and their copolymers as well as hyaluronic acid. The polymers may be used in pure form, in derivatized form, in the form of blends or as copolymers, depending on the desired properties. Metallic biodegradable materials are based on alloys of magnesium, iron, zinc and/or tungsten.

Stents are also known, which have coatings with various functions. Coatings of this type are used, for example, to release medications, to arrange x-ray markers or to protect the structures lying beneath.

In the realization of biodegradable implants, the degradability should be controlled according to the planned therapy or the use of the respective implant (coronary, intracranial, renal, etc.). For some therapeutic applications, the implant is designed to lose its integrity within a period of more than six months. The term “integrity,” i.e., mechanical integrity, hereby refers to the property that the implant undergoes hardly any mechanical losses in comparison with the undegraded stent. This means that the implant is still stable enough mechanically that, for example, the collapse pressure drops only slightly, i.e., at most to 80% of the nominal value. The implant can thus still fulfill its main function, namely keeping the blood vessel open, while the integrity of the stent is preserved. As an alternative, integrity may be defined such that the implant is so stable mechanically that it is subject to hardly any geometric changes in its load state in the vessel, for example, it does not collapse to any appreciable extent, i.e., under load it has at least 80% of the dilatation diameter, or in the case of a stent has hardly any supporting struts that have been broken through.

Biodegradable magnesium implants, in particular magnesium stents, have proven to be especially promising for the aforementioned target corridor of degradation, although on the one hand they lose their mechanical integrity or supporting effect too soon, and on the other hand, they have a highly fluctuating loss of integrity in vitro and in vivo. This means that in the case of magnesium stents the collapse pressure drops too rapidly over time or the drop in collapse pressure is too variable and is, therefore, indeterminable.

Various mechanisms for the degradation control of magnesium implants have already been described in the prior art. These are based, for example, on inorganic or organic protective layers or the combination thereof, which offer resistance to the human corrosion environment and the corrosion processes occurring there. Previously known solutions are characterized in that barrier layer effects are obtained, which are based on a spatial separation as free from defects as possible of the corrosion medium from the metallic material, in particular the metallic magnesium. For example, the degradation protection is ensured through various combined protective layers and through defined geometric distances (diffusion barriers) between the corrosion medium and the magnesium base material. Other solutions are based on alloying constituents of the biodegradable material of the implant body, which influence the corrosion process by shifting the position in the electrochemical series. Further solutions in the field of controlled degradation trigger predetermined breaking point effects through the application of physical (e.g., local changes in cross section) and/or chemical changes in the stent surface (e.g., locally chemically differently combined multilayers). However, it is generally not possible with the aforementioned solutions to place the dissolution occurring through the degradation process and the resulting strut breakages in the necessary time window. The result is that degradation of the implant occurs too soon or too late or is too variable.

Another problem in connection with coatings is due to the fact that stents or other implants usually assume two states, namely a compressed state with a small diameter and an expanded state with a larger diameter. In the compressed state the implant can be inserted into the vessel to be supported and positioned at the site to be treated by means of a catheter. At the site of treatment, the implant is then dilated by means of a balloon catheter, for example, or (when a shape-memory alloy is used as the implant material) converted to the expanded state, for example, by heating it to a temperature above the transition temperature. Due to this change in diameter, the body of the implant is hereby subjected to a mechanical stress. Additional mechanical stresses on the implant can occur during the manufacture or during the movement of the implant in or with the vessel in which the implant is inserted. With the known coatings, this thus results in the disadvantage that the coating tears during the deformation of the implant (e.g., formation of microcracks) or is also partially removed in large areas. Nonspecific local degradation can be caused hereby. Moreover, the onset and the speed of degradation depend on the size and the distribution of the microcracks forming due to the deformation, which are difficult to monitor as defects. This leads to a greater scattering in the degradation times. From printed publication DE 10 2006 060 501 a method is known for producing a corrosion-inhibiting coating on an implant of a biocorrodible magnesium alloy and an implant that can be obtained according to the method, in which after the implant has been provided, the implant surface is treated using an aqueous or alcoholic conversion solution containing one or more ions selected from the group comprising K⁺, Na⁺, NH₄⁺, Ca²⁺, Mg²⁺, Zn²⁺, Ti⁴⁺, Zr⁴⁺, Ce³⁺, Ce⁴⁺, PO₃³⁻, PO₄³⁻, HPO₄²⁻, H₂PO₄⁻, OH⁻, BO₃³⁻, B₄O₇³⁻, SiO₃²⁻, MnO₄²⁻, MnO₄⁻, VO₃⁻, WO₄²⁻, MoO₄²⁻, TiO₃²⁻, Se²⁻, ZrO₃²⁻, and NbO₄⁻, wherein a concentration of the ion or the ions is in the range of from 10⁻² mol/l to 2 mol/l, respectively. The treatment of the implant surface with the referenced conversion solution causes an anodic oxidation of the implant. It is carried out either without the use of an external power source (externally unpowered) or with a power source. However, the method examples and electrolyte compositions described in this publication do not meet the expectations in terms of degradation behavior and dilatation capability without layer destruction in the application for a magnesium stent.

From printed publication US 2008/0086195 A1 a medical device such as a catheter or a stent is known, in which a polymer-free coating is applied using a plasma electrolytic deposition process (PED). The plasma electrolytic coating hereby comprises a plasma electrolytic oxidation (PEO), a micro-arc oxidation (MAO), a plasma-arch oxidation (PAO), an anodic spark oxidation or a plasma electrolytic saturation (PES). The plasma electrolytic coating is carried out by means of pulsed AC voltage or DC voltage at voltages between −100 V to 600 V. The current densities are in the range of 0.5 to 30 A/dm². The known plasma electrolytic coating is provided in order to be able to incorporate into the coating additional active ingredients that contain a medication or another therapeutic agent. This printed publication therefore does not deal with the above problem.

### SUMMARY OF THE INVENTION

A feature of the present invention is therefore to disclose a method for producing an implant, which leads to an extension of the degradation time of the implant to a period of greater than five months. Moreover, the object is to create an implant with a correspondingly long degradation time.

The above feature is attained with a method in which the following steps are carried out:
a) Provide the body of the implant,
b) Apply a first layer, which contains Ca ions and P ions, to at least a part of the surface of the body and
c) Apply a second layer which is at least partially permeable for Ca ions and P ions, such that this at least largely covers the first layer, i.e., covers the majority of the surface of the first layer.

The first layer can hereby be applied directly to the surface of the implant body as well as to a coating arranged on the surface of the body (see below, for example).

The present invention utilizes the realization that the degradation of metallic alloys, in particular of Mg alloys, in the use, e.g., as a vascular implant, or as a biocorrodible implant in osteosynthesis, is associated among other things with the formation of CaP (calcium phosphate)-containing corrosion-product layers. These can achieve layer thicknesses that, depending on the dwell time in the body, are up to several micrometers and have a high proportion of the former initial cross section of the purely metallic implant. Due to their microcracking, however, these layers do not themselves represent a corrosion-protective system with self-healing properties, such as are observed, for example, in the protection system containing Cr⁶⁻ ions used in technology. CaP layers exhibit a low plastic deformation capability, which leads to a comparatively rapid fragmentation. The fragments are detached from the metallic base material and then expose it to corrosive attack again.

If an “artificial” calcium phosphate layer is now produced by means of the first layer according to the invention, in addition to the stoichiometric composition, this layer also has free or differently bonded Ca ions and P ions. Through the presence of these ions and the permeability of the second layer for these ions, the “natural” CaP layer forming in the body environment as a result of the degradation process can form more quickly through the reservoir of free Ca ions and P ions. The surface of the implant thus undergoes a “self-healing effect,” which leads to an additional cover layer formation. This cover layer thus represents an endogenous reaction product/corrosion product and seals the artificially produced CaP layer lying beneath at least for a limited period of time that can be predetermined. This leads to a shift of the loss of integrity into a period of greater than five months.

In a preferred exemplary embodiment, the first layer is applied by means of a plasma chemical method or by means of sand blasting. The cited methods are very suitable in particular for the method according to the invention because the composition of the first layer and thus the degradation behavior can be controlled very well through the composition of the electrolytes or the solids applied through the sand blasting.

After the application of the first layer, the implant is rinsed several times preferably in distilled water and thereafter immediately dried under hot air at a temperature of approx. 50° C. to approx. 80° C.

In a further preferred exemplary embodiment, before the application of the first layer, a third layer is applied containing hydroxides and/or oxides and/or fluorides of the base material of the body. The advantage of this third layer is that the entire layer system has a particularly high damage tolerance. For example, the mechanical loads on the stent surface occurring during the later handling of the implant (e.g., bending stress during the crimping of a stent) do not product any cracks that reach as far as the metallic base material due to the presence of the third layer.

It is furthermore advantageous if the body of the implant is pickled before the application of the first layer. Through the treatment with the pickling solution, for example with 20% alcoholic phosphoric acid, which is at room temperature, over a period of approx. 10 seconds to approx. 2 minutes, the residues (surface contaminants) on the implant body are removed up to a depth of greater than 2 μm. The pickling process also allows only very few atomic hydrogen ions to form, which can lead to a hydrogen embrittlement of the base material. In addition an electropolishing can be carried out before the application of the first layer and after the pickling of the implant body.

The second layer is particularly preferably applied by means of CVD (Chemical Vapor Deposition), PVD (Physical Vapor Deposition) or plasma polymerization. These methods are suitable for the production of the second layer because particularly thin layers can be applied therewith.

The layer system produced with the production method according to the invention has a high damage tolerance, a high plastic deformation capability and a high adhesion between the individual layers and to the body. The high plastic deformation capability is ensured in that any microcracks occurring are absorbed by the pore structure of the layers. The layers furthermore have a high biocompatibility, because in their chemical composition they resemble as far as possible the layers forming in the body environment. Moreover, the development of quantities of corrosion products that are problematic from a cytotoxic point of view is minimized, and at most noncritical inflammatory side effects occur in the surrounding biological tissue.

The above object is further attained through an implant which is provided with a first layer, which contains Ca ions and P ions, arranged on at least a part of the surface of the body and a second layer, which is permeable to Ca ions and P ions at least in part, such that the second layer at least to a large extent covers the first layer, i.e., covers the majority of the surface of the first layer.

The degradation mechanism/corrosion mechanism of the implant according to the invention is explained below, which leads to a degradation in the desired time window. With an implant according to the invention of this type, as explained above, the first layer, which represents an “artificial” CaP layer, lying beneath the second layer after implantation at the treatment site ensures that Ca ions and P ions are quickly available for the development of a “naturally” occurring CaP layer lying outside. The exchange of Ca ions and P ions hereby occurs through the second layer, which is permeable to these ions. Through the exchange of the Ca ions and the P ions through the second layer, initially a locally limited self-healing of the microholes present in the second layer is achieved. After a further progression of the corrosion, an intensified local exchange of Ca ions and P ions of the first layer and the “natural” CaP layer lying outside through the second layer takes place. Initially the self-healing effects and the corrosion are still hereby in the chemical equilibrium and the ions of the two CaP layers are mixed with one another. Thereafter the degradation of the first layer also begins, wherein the base material is not affected by the degradation. In the meantime the corrosion of the second layer progresses further and a self-healing is no longer guaranteed. The second layer now has numerous holes, in which the Ca ions and the P ions of the first layer and the “natural” CaP layer have mixed. With the further progression of the degradation, the weak points in the first layer extend up to the base material of the implant, the degradation of which now likewise begins. Now the ion exchange takes place over the entire cross section of the coating system and the degradation is accelerated.

In a preferred exemplary embodiment, the second layer contains Parylenes. In particular with a layer thickness between approximately 1 μm and approximately 3 μm, a Parylene layer exhibits a particularly advantageous behavior during the degradation. Particularly in the range of these layer thicknesses, the Parylene layer is permeable to Ca ions and P ions and is moreover so flexible that it also renders possible a dilatation of an implant. The Parylene C variant is used particularly preferably, wherein, however, a coating containing Parylene N can also be used.

In a further preferred exemplary embodiment, the layer thickness of the first layer is between approximately 0.5 μm and approximately 10 μm. The layer thickness range according to the invention is advantageous, since on the one hand it still represents an effective diffusion barrier for the corrosive medium and on the other hand (through the upper limitation of the layer thickness) still guarantees a minimum level of plastic deformation capability. Larger layer thicknesses can cause cracks and layer delaminations during dilatation.

In a further preferred exemplary embodiment, a third layer is arranged under the first layer on the body surface, which third layer has hydroxides and/or oxides and/or fluorides of the base material of the body and has a layer thickness of preferably less than approximately 150 nm. The advantages of the third layer have already been explained above. The third layer can be omitted in particular when the duration of degradation—depending on the indication—is to be adjusted such that it lies at the lower end of the degradation time of the implant according to the invention.

The method according to the invention or the implant according to the invention are explained below on the basis of examples and figures. All of the features described and/or shown thereby form the subject matter of the invention, regardless of their summary in the claims or their relation.

### DESCRIPTION OF THE DRAWINGS

The drawings show diagrammatically:

FIG. 1 A cross section through a part of a body of a first exemplary embodiment of an implant according to the invention and

FIG. 2 A cross section through a part of a body of a second exemplary embodiment of an implant according to the invention.

### DETAILED DESCRIPTION OF THE INVENTION

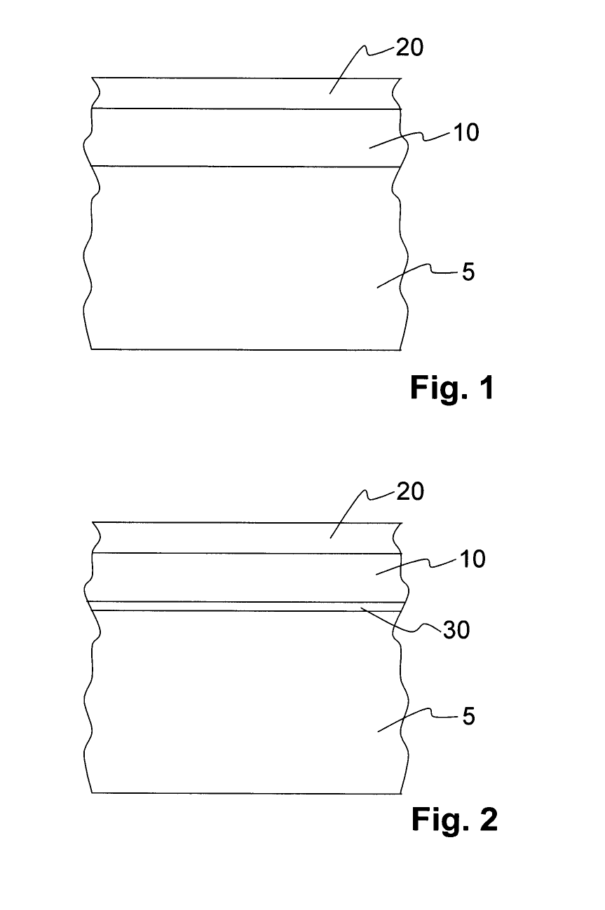

FIG. 1 shows a cross section of a part of the body of a first exemplary embodiment of an implant according to the invention. The implant is a stent, for example. A first layer 10 is arranged on the surface of the body 5, which first layer contains Ca ions and P ions and preferably has a layer thickness of between approximately 0.5 μm and approximately 10 μm.

The first layer 10 is composed, for example, of the following components: (in decreasing order)
Magnesium phosphate (30-50% by weight)
Calcium phosphate (30-40% by weight)
Magnesium oxide (15-20% by weight)
Magnesium carbonate (10-15% by weight)
Magnesium hydroxide (10-15% by weight)
Remainder (<5% by weight): non-stoichiometric potassium and sodium compounds

The first layer 10 can be applied, for example, by means of a plasma chemical method at voltages between 250 and 500 V, current densities between 0.5 and 5 A/dm², pulse frequencies between 100 Hz and 10 kHz and using an anticathode of stainless steel 1.4301 by treating the implant base body in an aqueous electrolyte of the following composition:
65 ml/l ethylenediamine solution (99%),
80 g/l potassium dihydrogenphosphate,
20 ml/l aqueous ammonium hydroxide solution (25%)
25 g/l sodium carbonate.

A second layer 20 lies above the first layer 10, which second layer is preferably composed of Parylene C and has a layer thickness of between approximately 1 μm and approximately 3 μm. The stents to be treated are placed in a coating changer. The coating process is started with a weighed-in quantity taking into consideration the chamber volume and the stent surface (e.g., approximately 4 g with 1 μm and 10 g with 3 μm layer thickness). The evaporator temperature is thereby between 100° C. and 170° C. The powdery monomer is suctioned via a heater plat based on the applied chamber volume of approx. 0.5 to 50 Pa e. The temperature of the heater plate is thereby between 650° C. and 730° C. After a coating period of approximately 1 hour with 1 μm (3 hours with 3 μm), the stents have a homogenous covering with Parylene C.

FIG. 2 shows a second exemplary embodiment of an implant according to the invention, in which, compared to the first exemplary embodiment, a third layer 30 is provided in addition below the first layer 10, which third layer has hydroxides and/or oxides and/or fluorides of the base material of the body 5 and has a layer thickness of preferably less than approximately 150 nm.

To produce a third layer 30 containing hydroxides of the base body, the body 5 of the implant is treated as follows:

Immersion at a temperature of 100° C. to 120° C. in aqueous NaOH solution (100 g/l to 200 g/l) for 5 to 10 minutes, subsequently three-fold rinsing in warm distilled H₂O and air dry at a temperature of 50° C. to 80° C.

Alternatively or additionally, the body 5 can be treated by means of an oxidation as follows, so that the third layer 30 has oxides of the base material of the body 5:

Deposit in a vacuum chamber, evacuate to approx. 0.1 mbar and ignite an oxygen plasma at a partial pressure of oxygen of approx. 1 mbar, treatment duration 10 minutes to 15 minutes.

Alternatively or additionally, the body 5 can also be fluorinated as follows so that the third layer 30 has fluorides of the base material of the body 5:

Dip the body 5 in 40% HF at room temperature over a period of 2 hours to 48 hours, subsequently rinse once in warm distilled H₂O, brief neutralization in aqueous NaOH solution (20 g NaOH/1) and subsequent 3-fold rinsing in warm distilled H₂O and air dry at a temperature of 50° C. to 80° C.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

### LIST OF REFERENCE NUMBERS

5 Body of the implant
10 First layer
20 Second layer
30 Third layer

1. A method for producing an implant, in particular an intraluminal endoprosthesis, wherein the base material of the body of the implant has biodegradable metallic material comprising Mg or an Mg alloy, the method comprising the following steps:
a. Provide the body of the implant,
b. Apply a first layer, which contains Ca ions and P ions, to at least a part of the surface of the body and
c. Apply a second layer, which is at least partially permeable for Ca ions and P ions, to at least largely covers the first layer.

2. A method according to claim 1, characterized in that the first layer is applied to the body by one of a plasma chemical method or by sand blasting.

3. A method according to claim 1, characterized in that before the application of the first layer to at least a part of the surface of the body, a third layer is applied containing one or more of hydroxides, oxides and fluorides of the base material of the body.

4. A method according to claim 1, characterized in that the body of the implant is pickled before the application of the first layer.

5. A method according to claim 1, characterized in that the second layer is applied by one or more of CVD, PVD or plasma polymerization.

6. An implant, in particular an intraluminal endoprosthesis, wherein the base material of the body of the implant has biodegradable metallic material comprising Mg or an Mg alloy, with a first layer, which contains Ca ions and P ions, arranged on at least a part of the surface of the body and a second layer, which is permeable to Ca ions and P ions at least in part, such that the second layer at least to a large extent covers the first layer.

7. An implant according to claim 6, characterized in that the second layer contains Parylenes.

8. An implant according to claim 6, characterized in that the first layer has a layer thickness between approximately 0.5 μm and approximately 10 μm.

9. An implant according to claim 6, characterized in that the second layer has a layer thickness between approximately 1 μm and approximately 3 μm.

10. An implant according to claim 6, characterized in that a third layer is arranged under the first layer on the surface of the body, which third layer has one or more of hydroxides, oxides and fluorides of the base material and has a layer thickness of less than approximately 150 nm.

11. An intraluminal endoprosthesis implant comprising:
a body having a surface and a base material including a biodegradable material including one or more of Mg and an Mg alloy;
a first layer having a thickness of between approximately 0.5 μm and approximately 10 μm, the first layer containing Ca ions and P ions and being arranged on at least a portion of the body surface;
a second layer having a thickness between approximately 1 μm and approximately 3 μm at least partially permeable to Ca ions and P ions, the second layer comprising parylenes, the second layer covering at least a portion of the first layer.

12. An intraluminal endoprosthesis implant as defined by claim 11 and further comprising a third layer arranged under the first layer on the body surface and having one or more of hydroxides, oxides and fluorides of the base material, the third layer having a thickness of less than approximately 150 nm.
